(19) United States
(12) Patent Application Publication
(10) Pub. No.: US 2003/0068822 A1
(43) Pub. Date: Apr. 10, 2003

(54) Hemoglobin assay calibration and control

(76) Inventors: Merrit Jacobs, Foxboro, NY (US); Karen Warren, Rush, NY (US); James E. Love JR., Penfield, NY (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 09/966,455

(22) Filed: Sep. 28, 2001

(51) Int. Cl. G01N 33/96

(52) U.S. Cl. 436/15

(57) ABSTRACT

Methods of calibrating and validating the calibration of hemoglobin assays include the use of calibrators and/or controls that incorporate cross linked blood substitute. The calibrators and controls can be formulated into kits.

### BACKGROUND

The invention relates to the calibration and control of assays for hemoglobin including assays for the determination of hemolized body fluids.

Many conventional clinical assays rely on well-established procedures by which standard curves relate analyte concentration or activity to some spectrophotometric measurement such as absorbance, transmission, or reflectance. This can be done by taking advantage of reactions with reagents that produce a detectable result such as color change but it is also possible to use this method in reagentless systems using, for example, near infrared radiation. Typically, spectrophotometric measurements are taken of solutions containing known analyte concentrations that span the dynamic range for that analyte. A curve is then fit to the data that can then be used to relate concentration of unknown samples to their spectrophotometric properties. The curve is typically validated from time to time by comparing concentrations arrived at by use of the standard curve against solutions having known concentrations. Such solutions of known concentration are referred to as controls. If the computed concentration values of the controls do not correspond sufficiently to the known values then the instrument used is recalibrated.

Controls and calibrators must have substantially similar spectrophotometric characteristics over the same concentration range one would expect to find in actual samples. This is often achieved by using aliquots of the same analyte for which the assay is conducted. Unfortunately, these analytes are sometimes not stable making controls and calibrators made from them unreliable. Hemoglobin is one such analyte. It gradually converts to methemoglobin over time which has a different spectra than does hemoglobin. One could use a dye that mimics the spectra of hemaglobin but thus far such dyes have only been found to simulate hemoglobin in portions of the spectra.

Hemoglobin is an important analyte in its own right but it can also be an important determinant of sample integrity. Sometimes it is found that samples of body fluids such as serum and plasma contain the contents of ruptured erythrocytes. Hemoglobin, being a red substance, can then color the sample to such a degree that the reliability of assays for other analytes cannot be assured. Such samples are referred to as hemolyzed samples. Clinicians typically are interested in determining whether samples are hemolyzed so that they can judge the reliability or integrity of the results obtained from them.

### SUMMARY OF THE INVENTION

In one aspect of the invention a method of calibrating hemoglobin assays includes the use of a cross linked blood substitute in a calibration fluid.

In another aspect of the invention a method of validating the accuracy or precision of hemoglobin assays includes the use of a cross linked blood substitute in a control fluid.

In yet another aspect of the invention calibration fluids are prepared from aliquots of a cross linked blood substitute. The aliquots have known concentrations that preferably span the dynamic range of the assay. Kits include the calibrator standards.

In yet another aspect of the invention control fluids are prepared from aliquots of a cross linked blood substitute. The aliquots have known concentrations that preferably lie within the dynamic range of the assay. Kits include the control fluids.

### BRIEF DESCRIPTION OF THE DRAWINGS

FIG. 1 is a multi-frequency plot of the absorbance spectra of cross linked blood substitute.

FIG. 2 is a plot of the concentration of refrigerated cross linked blood substitute of the instant invention determined over the course of twenty days using a sample spiked at 10 g/L.

FIG. 3 is a plot of the concentration of refrigerated cross linked blood substitute of the instant invention determined over the course of twenty days using a sample spiked at 6.5 g/L.

FIG. 4 is a plot of the concentration of refrigerated cross linked blood substitute of the instant invention determined over the course of twenty days using a sample spiked at 4.33 g/L.

FIG. 5 is a plot of the concentration of refrigerated cross linked blood substitute of the instant invention determined over the course of twenty days using a sample spiked at 1.3 g/L.

### DETAILED DESCRIPTION

The most preferred cross linked blood substitute of the instant invention is a commercially available product sold under the tradename “OXYGLOBIN” solution by Biopure Corporation (Cambridge, Mass.). One skilled in the art will appreciate that other cross linked blood substitutes having similar spectrophotometric characteristics are also suitable for use in the instant invention. The “OXYGLOBIN” substitute is made by fractionating blood raw product (which may be non-human such as bovine or porcine blood) to produce a red blood cell fraction, disrupting the red blood cell fraction to produce a hemoglobin-containing solution, conducting various filterations, and cross linking the hemoglobin solution. Cross linking is accomplished through the use of a cross linking agent such as glutaraldehyde. The cross linked blood substitute and the method of making it are more fully described in U.S. Pat. Nos. 5,084,558; 5,296,465; 5,618,919; 5,691,452 (all assigned to Biopure Corporation) each of which is incorporated herein by reference.

Calibrators are made by preparing aliquots of different concentrations of the cross linked blood substitute. This is done according to well-known methods. The best method involves starting with a known concentration and preparing calibration standards from serial dilutions. The dynamic range of hemaglobin extends from about 0.2 g/dl to about 8.0 g/dl. Preferably, a five point curve is prepared but standard curves made from more or fewer points are acceptable. The number and concentration of standards depends upon the application and is readily determined by routine experimentation. Standards concentrations go from 0.2 g/dl to 8.0 g/dl, The diluent can be any of the well-known diluents used to make calibration standards. Preferably, it is is a buffered diluent or deionized water. OXYGLOBIN solution from Biopure is commercially available at a concentration of 13 g/dl of the cross linked blood substitute. One skilled in the art will readily appreciate the quantities of diluent necessary to constitute the standards. Optionally, liquid calibrators that do not require a diluent can also be prepared as can lyophilized calibrators.

Controls are made in the same manner as the calibrators. A two (high, low concentration) or three point (high, medium, low) control package is preferred but any number of controls can be used. The two point controls typically comprise a known low concentration control of between 0.5 g/dl and 1.0 g/dl of the cross linked blood substitute and a known high concentration control of between 2.0 g/dl and 4.0 g/dl of the cross linked blood substitute. Buffers and/or stabilizers are optionally added to the calibration standards according to well-known methods. The control and/or calibration compositions can include still other analytes, particularly if so-called “universal” calibrators or controls are desired.

The controls and calibrators of this invention are suitable for quality assurance purposes when they have concentrations a) that are suitable for establishing a calibration curve (in the case of calibrators) for determining the quantity of hemoglobin in an unknown across the expected range of concentrations in the animal in question (preferably in humans) and b) that are suitable for determining the validity of a calibration curve or measurement, or assay protocol (in the case of controls). The controls are used to ensure sufficient precision and accuracy is evident in the analytical processes. Generally, this occurs when test to test coefficients of variation are no greater than about 15% in the case of precision. Accuracy is suffient when the variance between the actual concentration of a control differs from the value attained through the conduct of the anlaysis by no more than about 8%.

The kits of this invention include calibration standards, controls, or both. Preferably, they also include a plurality of containers or vessels and appropriate instructions.

The controls and/or calibrators of the instant invention can be used to good advantage in sample integrity applications. In such applications, the sample is interrogated to determine whether the sample contains too great a concentration of a substance (e.g., hemoglobin) that could interfere with the accuracy or precision of assays conducted on the sample. Preferably, such determinations are conducted on the sample before general testing begins. This allows the operator to be signaled that the sample is, for example, hemolyzed. This enables the operator to abort further testing or flag the results for further consideration. The most preferred method of conducting this type of analysis is described in U.S. Pat. No. 6,013,528 assigned to Ortho-Clinical Diagnostics, incorporated herein by reference. When using such an application, the controls of this invention can periodically be placed in sample vessels on the analyzer. The control sample is then subjected to sample integrity testing as would a patient sample. The results of the analysis are then used to determine whether the sample integrity testing is being accurately and/or precisely performed or whether corrective action is necessary. Other methods for conducting sample integrity evaluations are well known and can also be used with the calibrators and/or controls of this invention.

The following non-limiting examples further illustrate the invention.

### EXAMPLE 1

(Spectra Correlation)

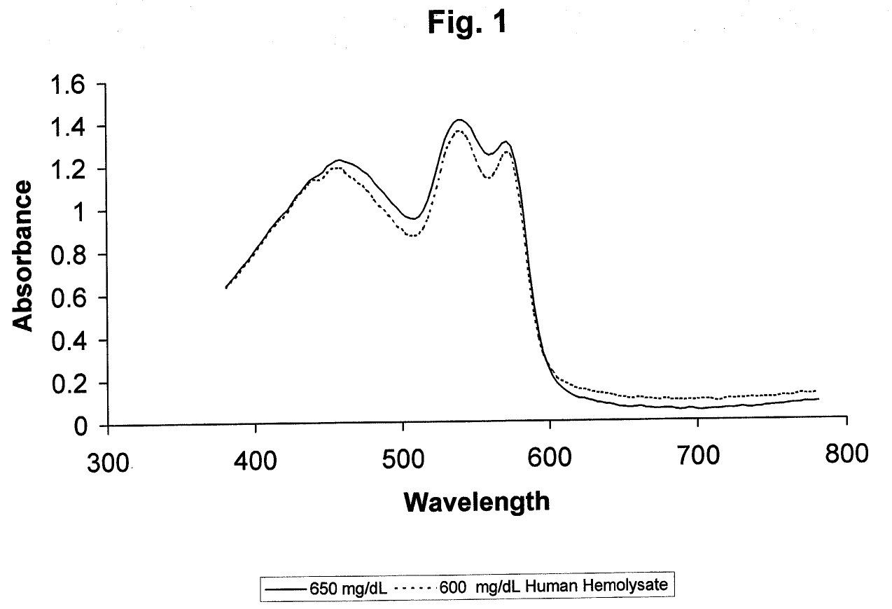

0.133 ml of Oxyglobin cross linked blood substitute (concentration of 13 g/L), obtained from Biopure Corporation (Boston, Mass.), was placed in a_{—}4.0 ml volumetric flask and qs to 4.0 ml with a clear human serum pool (no visual hemolysis. A similar sample was made up using 0.369 mls of a prepared human hemolysate (concentration of 4.7 g/L) was placed in a_{—}4.0 ml volumetric flask and qs to 4.0 ml with a clear human serum pool (no visual hemolysis The samples were subjected to spectrophotometric analysis using a module with a MicroParts VIS spectrometer at wavelengths of 400-800 nm. The absorbance spectra from the analysis is shown in FIG. 1. Spectral scans overlay on top of each other with the same absorbance peaks at the same wavelength. This illustrates that the cross linked blood substitute used in the instant invention provides acceptable performance as both a calibration fluid and control fluid. That is, a spectrophotometric determination of the concentration of the cross linked blood substitute is predicative of the concentration of human hemoglobin.

### EXAMPLE 2

(Controls)

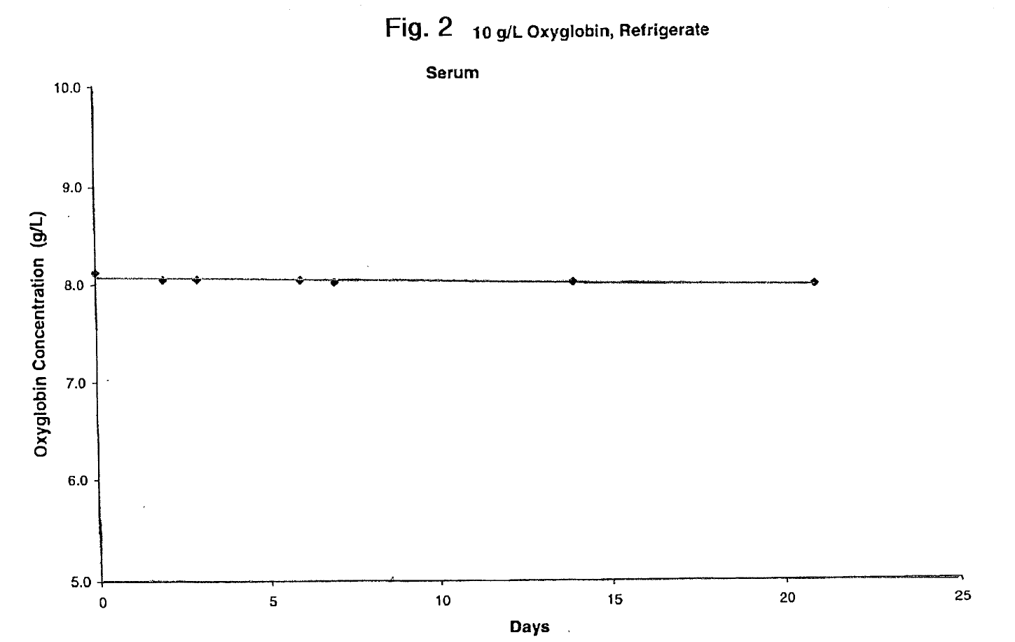

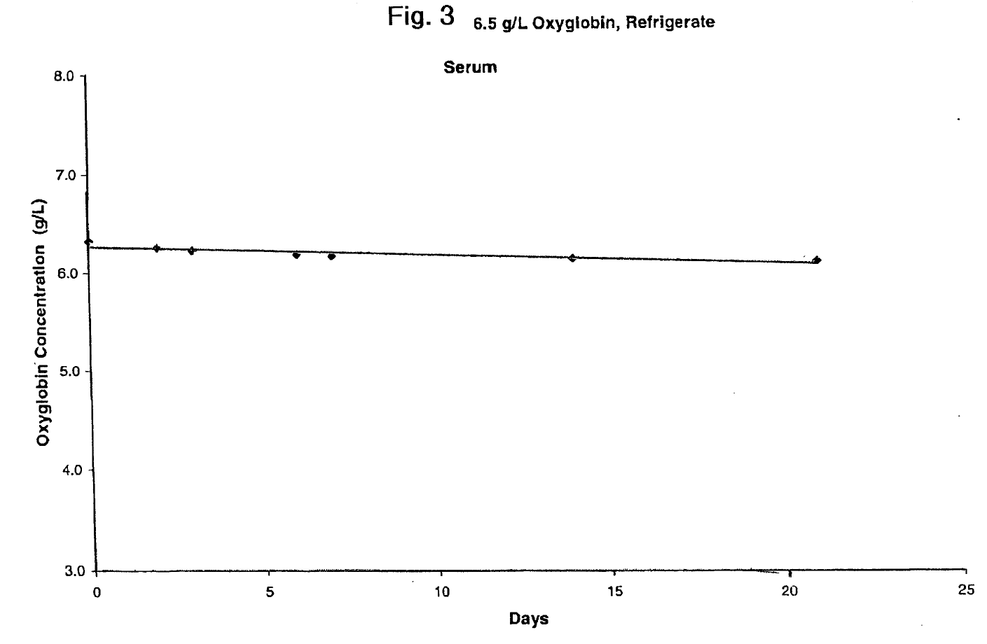

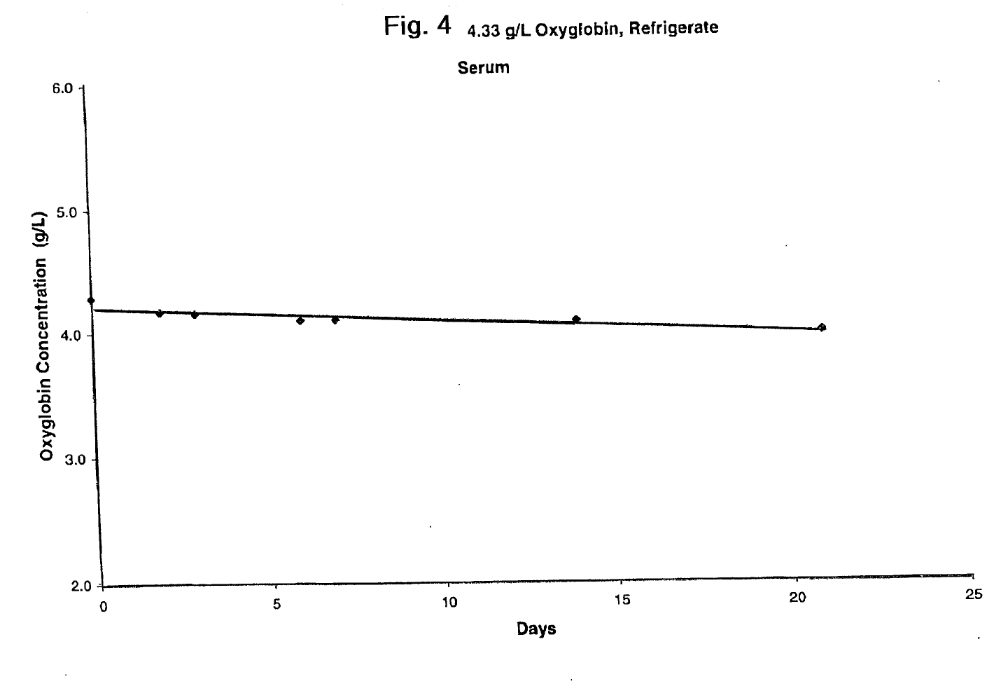

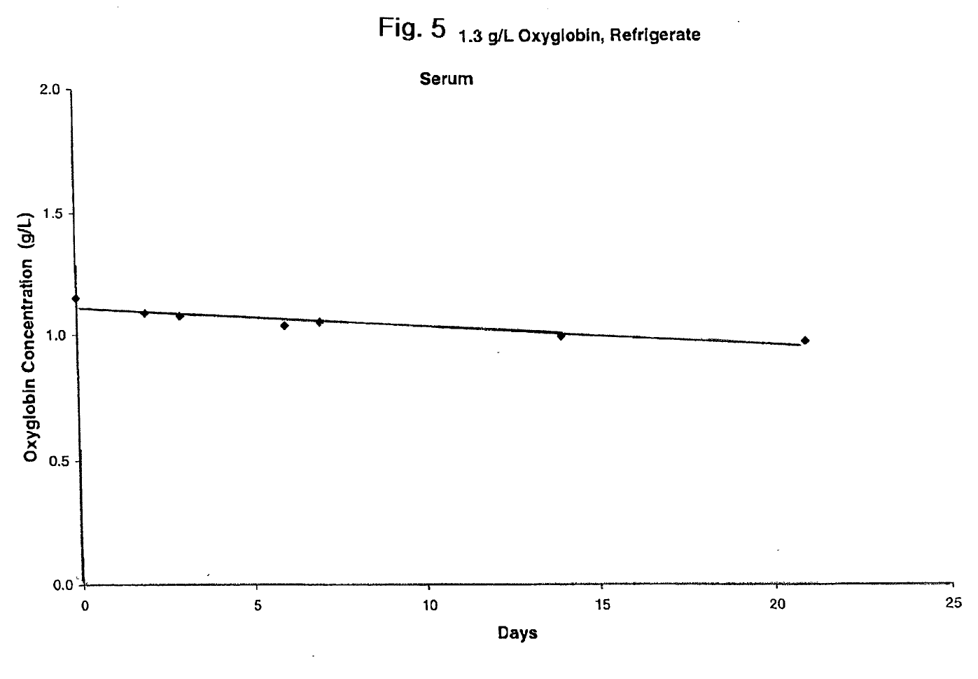

The cross linked blood substitute of Example 1 was diluted with a clear human serum pool to produce aliquots containing 1 g/L, 6.5 g/L, 4.33 g/L, and 1.3 g/L. Each aliquot was subjected to spectrophotometric analysis using a module with a MicroParts VIS spectrometer to determine concentration. Samples were then refrigerated to 2- 8° C. Analyses were again conducted three days after preparation, four days after preparation, six days after preparation, seven days after preparation, fourteen days after preparation, and twenty one days after preparation. The results are shown in FIGS. 2-5 respectively.

This example illustrates that refrigerated cross linked blood substitute can be used reliably as a control material for human hemoglobin assays. Concentration determinations made using non-refrigerated samples did not retain stability over the same period largely due to the transformation of hemoglobin to methemoglobin. This is a well known phenomena in non-refrigerated human hemoglobin as well.

### EXAMPLE 3

(Sample Integrity)

Cross linked blood substitute of Example 1 was diluted according to Table 1. Aliquots were stored in small vials with caps at 2-8° C. The “Through the Tip” analysis of aspirated samples described in U.S. Pat. No. 6,013,528 was conducted using 100 μl of sample in a sealed probe tip. The assays were conducted using a_module with a MicroParts VIS spectrometer. Aliquot concentrations were accurately determined for all concentrations. Over a three week period, refrigerated aliquots showed a slight decrease in hemoglobin concentration of about 4-13%. Non-refrigerated aliquots degrade to a greater extent.

This example illustrates that the cross linked blood substitute is suitable as a control fluid for sample integrity testing. It also illustrates that dilutions can reliably be made for establishing a calibration curve. Multiwavelength analyses can be conducted in such applications. The algorithms used to relate concentration to signal in such analyses require mathematical transformation (e.g., Fourier Transform).

TABLE 1

| Dilution Factor | Concentration (mg/dL) | Diluent |
|---|---|---|
| 13X | 1000 | Normal Human Serum Pool |
| 20X | 650 | Normal Human Serum Pool |
| 30X | 433 | Normal Human Serum Pool |
| 100X | 130 | Normal Human Serum Pool |
| 13X | 1000 | Pooled Bovine Serum |
| 20X | 650 | Pooled Bovine Serum |
| 30X | 433 | Pooled Bovine Serum |
| 100X | 130 | Pooled Bovine Serum |

We claim:

1. A process for making a clinical control, calibrator or standard for hemoglobin analysis, comprising employing cross linked blood substitute to produce solutions with known concentrations suitable for quality assurance purposes.

2. A process of claim 1 wherein said control, calibrator or standard is a single-component control, calibrator or standard.

3. A process of claim 1 wherein said control, calibrator or standard is a multi-component control, calibrator or standard.

4. The process of claim 1 further comprising combining the cross linked blood substitute with diluent.

5. The process of claim 1 wherein the cross linked blood substitute is refrigerated.

6. A method of calibrating an analyzer for conducting hemoglobin assays comprising preparing a calibration curve using a calibration standard comprised of a cross linked blood substitute.

7. The method of claim 6 wherein said calibration standard comprises 0.2-8 mg/dl of the cross linked blood substitute.

8. The method of claim 6 wherein the cross linked blood substitute is refrigerated.

9. A method of assessing the validity of hemoglobin analysis comprising conducting a hemoglobin assay using a control comprised of a known concentration of cross linked blood substitute to assess whether the analysis is sufficiently accurate or precise.

10. The method of claim 10 wherein said control comprises 0.5-4.0 mg/dl of the cross linked blood substitute.

11. The method of claim 9 used in sample integrity analysis.

12. A calibration kit comprising a solution of a known concentration of cross linked blood substitute.

13. The kit of claim 12 further comprising calibrator containers and instructions wherein the cross linked blood substitute is refrigerated.

14. A control kit comprising a solution of a known concentration of cross linked blood substitute.

15. The kit of claim 14 further comprising control containers and instructions wherein the cross linked blood substitute is refrigerated.
